(19) United States
(12) Patent Application Publication
(10) Pub. No.: US 2007/0079658 A1
(43) Pub. Date: Apr. 12, 2007

(54) Rotating aperture for ultrasound imaging with a capacitive membrane or electrostrictive ultrasound transducer

(75) Inventor: Paul A. Wagner, El Cerrito, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/356,886

(22) Filed: Feb. 17, 2006

Related U.S. Application Data
(60) Provisional application No. 60/719,810, filed on Sep. 23, 2005.

(51) Int. Cl. A61B 8/00; G01N 29/00

(52) U.S. Cl. 73/627; 600/437

(57) ABSTRACT

An aperture rotates for ultrasound imaging with an ultrasound transducer responsive to bias for operation, such as cMUTs or electrostrictive crystal transducers. By rotating a bias aperture relative to a time delay aperture, a more isotropic beam profile results. Acoustic energy is transmitted with one arrangement of bias and time delay apertures. The bias and/or time delay apertures are rotated for receiving acoustic energy in response to the transmitted acoustic energy. The two-way convolution of the different aperture positions results in a more isotropic beam profile.

### RELATED APPLICATIONS

The present patent document claims the benefit of the filing date under 35 U.S.C. §119(e) of Provisional U.S. Patent Application Ser. No. 60/719,810, filed Sep. 22, 2005, which is hereby incorporated by reference.

### BACKGROUND

The present embodiments relate to ultrasound imaging with a capacitive membrane or microfabricated ultrasound transducer (cMUT) or electrostrictive crystal transducer. cMUTs may be formed from semiconductor material or from other materials. A plurality of membranes or other structures with electrodes transduce between acoustic and electrical energies. Groups of the membranes operate as different elements. Various arrangements of elements may be provided on the cMUT, such as multi- or two-dimensional arrays of elements.

To operate a cMUT, the membranes are biased by a DC voltage. Alternating signals are applied to the elements to generate acoustic energy. Acoustic energy received by the elements is converted into alternating signals.

U.S. Patent Published Application No. 2004/0160144 shows a multidimensional cMUT. The bias voltages are applied as a Fresnel aperture. Positive, negative or zero level bias voltages are applied in a pattern to focus acoustic energy. The alternating signals are used for beamforming with a time delay aperture. The time delay and Fresnel apertures are orthogonal to each other. Higher side lobes may result from the Fresnel aperture focus than from the time delay aperture. For three-dimensional imaging, poor side lobes along one axis can negatively influence image quality in any slice orientation.

### BRIEF SUMMARY

By way of introduction, the preferred embodiments described below include methods, systems and improvements for ultrasound imaging with a capacitive membrane or electrostrictive ultrasound transducer. By rotating a bias aperture and a time delay aperture, a more isotropic beam profile results. Acoustic energy is transmitted with one arrangement of bias and time delay apertures. The bias and time delay apertures are rotated for receiving acoustic energy in response to the transmitted acoustic energy. The two-way convolution of the different aperture positions results in a more isotropic beam profile.

In a first aspect, a method is provided for ultrasound imaging with an ultrasound transducer responsive to a bias. Acoustic energy is transmitted from the ultrasound transducer with bias lines connected along a first direction of the ultrasound transducer and transmit signal lines connected along a second direction different from the first direction. Acoustic energy is received with the ultrasound transducer in response to the transmitting and with the bias lines connected along a direction different from the first direction and receive signal lines connected along a direction different from the second direction.

In a second aspect, an improvement in a method for transmitting and receiving acoustic energy with an ultrasound transducer responsive to a bias for transduction operation is provided. The transmitting and receiving are responsive to bias signals applied to the ultrasound transducer and to alternating signals. The improvement includes interchanging the bias signals and alternating signals between a transmit event and a receive event responsive to the transmit event.

In a third aspect, a further improvement in a method for transmitting and receiving acoustic energy with an ultrasound transducer is provided. The transmitting and receiving are responsive to bias signals, which may or may not fluctuate, and to alternating signals. The further improvement includes adjusting the bias pattern while receiving to dynamically focus in phase at a multiplicity of depths. The DC bias is changed slowly to prevent the unwanted generation of acoustic energy. Alternatively, such energy is generated and then filtered out by the imaging system.

In a fourth aspect, a system is provided for ultrasound imaging with an ultrasound transducer responsive to a bias for operation. First electrodes are on the ultrasound transducer. The first electrodes are distributed across a second direction and each extends over multiple elements along a first direction. Second electrodes are on the ultrasound transducer. The second electrodes are distributed across the first direction and each extends over multiple elements along the second direction. A bias generator is connectable with the first and second electrodes. Alternating signal lines are connectable with the first and second electrodes. At least one switch is operable to connect the bias generator to the first electrodes during transmit and the second electrodes during receive and operable to connect the alternating signal lines to the second electrodes during transmit and the first electrodes during receive.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a circuit diagram of one embodiment of a system for ultrasound imaging with a cMUT or electrostrictive crystal transducer;

FIG. 2 shows graphical representations of three embodiments of Fresnel apertures;

FIG. 3 is a graphical representation of a combination of two Fresnel apertures according to one embodiment;

FIG. 4 is a flow chart diagram of one embodiment of a method for ultrasound imaging with a cMUT or electrostrictive crystal transducer; and

FIG. 5 is a timing chart diagram for the method of FIG. 4.

### DETAILED DESCRIPTION OF THE DRAWINGS AND PRESENTLY PREFERRED EMBODIMENTS

The bias lines and alternating signal lines are interchanged between transmit and receive events. For example, orthogonal Fresnel bias and time delay axes are electronically interchangeable, effectively rotating the acoustic aperture by 90 degrees. By electronically swapping the Fresnel and time delay axes between transmit and receive, the round trip beam profiles in both azimuth and elevation are the product of both the Fresnel and time delay beam profiles. Side lobes may be further reduced by using seven or more quantized bias levels to apodize the Fresnel bias pattern. The beam profile can be made even more isotropic by dynamically adjusting the Fresnel bias pattern on receive. The transducer may have electrodes in a matrix configuration where N² element addressability is achieved with only 2N connections. Other embodiments with or without one or more of the features discussed above may be provided.

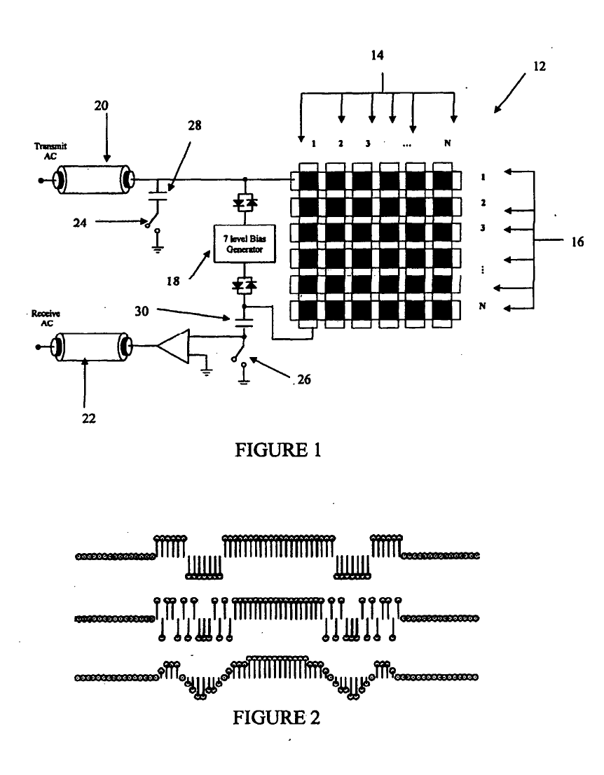

FIG. 1 shows a system for ultrasound imaging with a capacitive membrane or microfabricated ultrasound transducer 12 (herein referred to as a cMUT or capacitive membrane ultrasound transducer) or an electrostrictive crystal transducer 12. The system includes the transducer 12, a bias generator 18, transmit lines 20, receive lines 22, high voltage switches 24, low voltage switches 26, and grounding capacitors 28, 30. Additional, different or fewer components may be provided. For example, the transmit and receive lines 20, 22 are the same lines. As another example, switches directly connect or disconnect the transmit and receive lines 20, 22 and the bias generator 18 to different electrodes 14, 16 of the transducer 12.

The system is integrated on a substrate. For example, the bias generator 18, cMUT 12, and low voltage switches 26 are integrated on a same semiconductor substrate. Additional, different or fewer components may be integrated. A different substrate than used for the cMUT 12 or discrete components may be used for the components of the system. A separate crystal structure may be used, such as associated with electrostrictive transducers.

FIG. 1 shows a single channel. For operation of the transducer 12, a plurality of parallel channels is provided. Each channel corresponds to a receive or transmit beamformer channel and associated electrode 14, 16. Separate components are used for the different channels, such as separate transmit lines 20, receive lines 22, high voltage switches 24 and low voltage switches 26. Alternatively or additionally, one or more components are used for more than one channel. For example, the bias generator 18 is used for all of the channels or for more than one channel with a same bias level.

The transducer 12 is a cMUT or electrostrictive crystal transducer. The transducer is responsive to a bias for transduction operation. As an electrostrictive transducer, the transducer 12 includes a plurality of crystals patterned or diced into elements. As a cMUT, the transducer 12 includes a plurality of membranes or other structures and associated voids or chambers. The membranes are flexible. Electrodes 14, 16 are positioned within the chambers and on the membranes. A grounding electrode is provided in common for all the elements, such as a ground electrode positioned on an outer surface of the cMUT 12 as a membrane-connected electrode. The movement of the membrane and the corresponding potential differences between the electrodes 14, 16 and ground allows transduction between acoustical and electrical energies. The membranes are sized and shaped to operate at desired frequencies, such as any bandwidth centered at an ultrasound frequency (e.g., a frequency within the range of 1-20 MHz). The membranes, chambers and electrodes 14, 16 may be formed on a semiconductor substrate with CMOS or other microfabrication techniques.

The interconnections of the electrodes 14, 16 define a plurality of elements. The transducer 12 of FIG. 1 is a multidimensional (e.g., N×N) array of elements. One dimensional or other multidimensional distributions of elements in a rectangular, triangular, hexagonal, or other grid may be provided. Each element includes many membranes or other flexible structures, such as tens or hundreds of membranes and chambers, electrically connected in parallel. A greater or less number may be provided, such as a single membrane and chamber for each element.

The electrodes 14, 16 include interconnections between elements. The electrodes 16 connect to different groups of elements than the electrodes 14. The electrodes 14, 16 are in a matrix pattern with rows of electrodes 16 extending along elevation and columns of electrodes 14 extending along azimuth. 1 to N electrodes 16 are distributed across an azimuth direction, and each extends over multiple elements along the elevation direction. 1 to M electrodes 14 are distributed across the elevation direction, and each extends over multiple elements along the azimuth direction. In FIG. 1, the number of each type of electrodes 14, 16 are shown as equal (N), but unequal numbers may be used. Each electrode 14, 16 is one element wide, but wider electrodes 14, 16 may be used. The length of each electrode 14, 16 extends across the entire transducer 12, but shorter lengths may be provided, such as dividing one or more rows or columns into two or more electrodes 14, 16.

The electrodes 14, 16 are orthogonal to each other, such as being along azimuth and elevation dimensions. In alternative embodiments, the electrodes 14, 16 are at other angles across the transducer 12, such as 45 degrees. More than two sets of electrodes 14, 16 may be provided, such as three sets of electrodes in a matrix pattern across the transducer 12 at 60 degree angles to each other.

Both a DC bias on one electrode 14, 16 and signal information on the other electrode 16, 14 are applied to a same acoustic element. For example, both electrodes 14, 16 connect with the electrodes within the chambers of the elements. In one embodiment, the electrodes 14, 16 and cMUT disclosed in U.S. Patent Publication Nos. 2004/0160144 (application Ser. No. 10/367,106, filed Feb. 13, 2003) and U.S. patent application Ser. No. 10/819,094 filed Apr. 5, 2004, the disclosures of which are incorporated herein by reference, are used. In alternative embodiments, the electrodes 14 connect to element electrodes in the chamber, and the electrodes 16 connect to element electrodes on the membrane. Alternatively, both the bias voltage and signal information may be combined on the same electrode 14, 16. While shown as rectangular arrangements in FIG. 1, the electrodes 14, 16 may comprise traces, jumpers or other electrical interconnections.

The bias generator 18 is a high voltage FET network connected with a voltage source. Different transistors, switches, voltage dividers, transformers, voltage generators or other devices may be used. Any now known or later developed bias generator 18 may be used.

The bias generator 18 is connectable with both sets of electrodes 14, 16. In the embodiment of FIG. 1, the bias generator 18 connects with the electrodes 14, 16 through diodes. Ground connections provided by the switches 24, 26 alternately connect the bias generator 18 to a different electrode 14, 16 than used by the alternating signals. In alternative embodiments, a multiplexer or other circuit arrangement switchably connects the bias generator to the different sets of electrodes 14, 16. The bias generator 18 includes a sufficient number of outputs, either as discrete outputs or outputs connected to a fewer number of bias generator circuits, to connect with a maximum number of electrodes 14, 16 used in a bias aperture.

In one embodiment, the bias generator 18 generates alternating waveforms at a frequency less than the alternating frequency of operation of the transducer 12 (ultrasound frequency) to act substantially as a DC bias. A bias voltage frequency of less than or equal to ⅓ the frequency of the alternating signal may be “substantially DC.” For example, a 500 KHz waveform is generated. By switching at about 500 KHz, a sinusoidal waveform may be used to gradually increase and decrease the bias voltage between transmit and receive events. The gradual transition, such as over one or two microseconds, may avoid generation of undesired acoustic transmissions. Any unwanted sound generated during the transition may be filtered out from the received signal. At the substantially zero portion of the bias waveform, the bias generator 18 may be switched to another electrode 14, 16. At the bias waveform peaks, the connected electrode 14, 16 is biased for transmission or reception. Alternatively, gradual transition is provided by stepped DC transition or switching. In yet other alternative embodiments, the bias generator has no or a more rapid transition.

The bias generator 18 is operable to generate at least two different bias levels, such as a zero bias and a non-zero bias or negative and positive biases selected for a desired sensitivity of the transducer 12, such as 10-120 volts. With three bias levels or two non-zero levels, relative phasing may be used for a Fresnel focus. The bias generator 18 outputs positive, zero and negative voltages as the biases applied to one set of electrodes 14, 16. The opposite or 180 degree phase shift resulting from opposite polarity biases on different electrodes focuses the acoustic energy. At one or more focal regions, the acoustic energy phases align. A greater number of bias levels may be used, such as five or seven bias levels. Different bias levels are applied to different electrodes 14, 16, forming an apodized Fresnel aperture for use during transmit and/or receive events. Symmetrical or non-symmetrical bias levels (e.g., two positive and three negative levels) may be used. On receive, the bias levels and/or Fresnel bias pattern may smoothly fluctuate over the duration of the receive event in a way that dynamically focuses based on phase.

The number of phase changes along one side or half of the Fresnel aperture is the same as the number of cycles used in the excitation waveform. For example, the excitation waveforms are two or three cycles. A greater or less number of phase changes and cycles may be provided. A number of phase changes different than the number of cycles may be used. FIG. 2 shows, at the top graphical representation, a Fresnel aperture using three bias levels with three phase changes for each half of the Fresnel aperture.

A zero bias is applied to electrodes 14, 16 outside of an active region of the Fresnel aperture. The Fresnel apertures shown in FIG. 2 have zero bias applied outside of each active aperture. Alternatively, a rapidly alternating bias consisting of alternating regions of negative and positive bias may be used to cancel the signal waveforms for elements outside the active aperture.

To reduce sidelobes, the bias pattern is altered to have transitions that are more gradual. The more gradual effect is accomplished by inserting short sections of alternating bias (+−) into the region surrounding a bias phase transition, such as shown by the middle Fresnel aperture as compared to the top Fresnel aperture of FIG. 2. Alternatively, the Fresnel aperture is apodized with intermediate bias levels, such as shown by the lower Fresnel aperture of FIG. 2. The apodized Fresnel aperture shown in FIG. 2 has seven discrete bias levels evenly spaced over a positive maximum to a negative maximum of the same magnitude. Other distributions of levels may be used.

Sidelobe levels may alternatively or additionally be reduced (or main lobes narrowed) by apodizing differently as a function of time. For example, multiple Fresnel apertures are used, one for transmit operation and one for receive operation. Analytical apodized bias pattern for N firings are represented as:

$${V}_{k} ( x , z ) = sin [ ω \cdot ( {T}_{k} + \frac{\sqrt{{x}^{2} + {z}^{2}} - z}{{c}_{W}} ) ]$$

$${T}_{k} = \frac{π \cdot ( N - k )}{ω \cdot N} , k = 1 , 2 , 3 … N$$

For each Firing k, Tₖ is the delay in seconds added to the waveform before beam summation for one-way response. For two-way response, the number should be doubled.

For dynamic receive focus, the bias pattern is given as a function of time, where t=0 is the time of the transmit firing, as:

$${V}_{k} ( x , t ) = sin [ ω \cdot ( {T}_{k} + \sqrt{\frac{{x}^{2}}{{c}_{W}^{2}} + \frac{{t}^{2}}{4}} - \frac{t}{2} ) ]$$

Two apodized Fresnel firings, when added together, may generate more ideal phase across their aperture. Bias levels are assigned in a way that simultaneously minimizes both the phase error and the amplitude distortion of the reconstructed aperture. Additional improvement in sidelobes can be achieved with four firings, capturing the acoustic cross-terms between transmit and receive. Bias interleaving is implemented by first optimizing a pattern with 2N−1 available bias levels and doubling the bias line pitch. The apodization values are then back-projected onto groups of two or more normal bias lines. The ideal phase is represented as:

$${ϕ}_{PERFECT} = \frac{ω}{{c}_{W}} ( \sqrt{{x}^{2} + {z}^{2}} - z )$$

φ_{PERFECT} is inverted to create a defocused point source, providing:

$${V}_{F 1} ( t , x , z ) = cos [ \frac{ω}{{c}_{W}} ( \sqrt{{x}^{2} + {z}^{2}} - z ) ] \cdot {ⅇ}^{jω \cdot t} \cdot {ⅇ}^{- j \frac{π}{2}}$$

$${V}_{F 2} ( t , x , z ) = sin [ \frac{ω}{{c}_{W}} \cdot ( \sqrt{{x}^{2} + {z}^{2}} - z ) ] \cdot {ⅇ}^{jω \cdot t}$$

$${V}_{F 1} + {V}_{F 2} = {ⅇ}^{j ( ω \cdot t - \frac{π}{2} )} \cdot ( cos [ \frac{ω}{{c}_{W}} ( \sqrt{{x}^{2} + {z}^{2}} - z ) ] + j \cdot sin [ \frac{ω}{{c}_{W}} \cdot ( \sqrt{{x}^{2} + {z}^{2}} - z ) ] )$$

$${V}_{F 1} + {V}_{F 2} = {ⅇ}^{j ( ω \cdot t - \frac{π}{2} )} \cdot {ⅇ}^{j \frac{ω}{{c}_{W}} ( \sqrt{{x}^{2} + {z}^{2}} - z )}$$

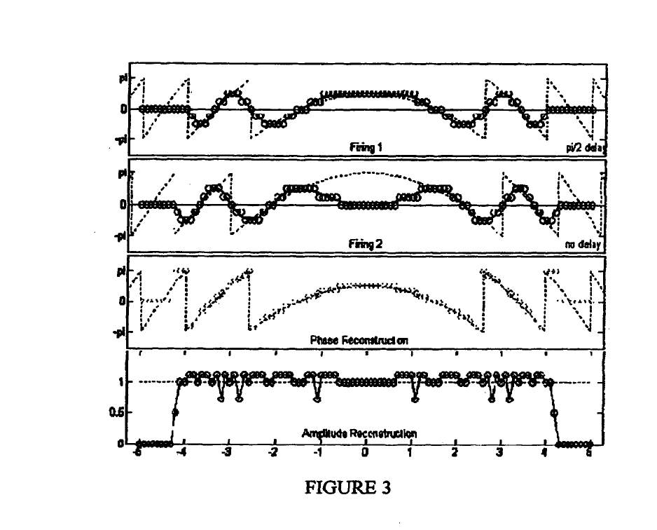

which is equal to φ_{PERFECT} plus an offset. FIG. 3 shows two Fresnel apertures for different transmit firings and the associated phase and amplitude reconstructions. The reconstructions correspond to combining received signals from a same scan line but associated with the two (or more) different transmit Fresnel apertures. Two or four back-to-back firings are used to interrogate the same region of the tissue. The difference between these firings is the Fresnel patterns used on transmit and receive.

An interleaved bias line pattern with N bias levels and W line width performs essentially as well as a non-interleaved bias pattern with 2N bias levels and 2W line width. The same degree of sidelobe reduction may be achieved by either apodizing more smoothly using wide elements, or by using coarser apodization that toggles frequently (interleaving) along a finer element pitch. In other words, doubling the number of available bias levels allows the total number of bias lines to be cut in half.

Referring again to FIG. 1, for transmit and receive events, the bias generator 18 applies bias sequentially to different sets of electrodes 14, 16, such as applying a Fresnel aperture in elevation for transmit and in azimuth for receive. The alternating signal lines 20, 22 are applied in an opposing manner, such as applying beamforming with the alternating signal lines in azimuth for transmit and in elevation for receive.

The alternating signal lines 20, 22 are traces, wires and/or coaxial cables. The alternating signal lines 20, 22 include separate transmit alternating signal lines 20 and receive alternating signal lines 22. Alternatively, at least a portion of the transmit and receive alternating signal lines 20, 22 share a same trace, wire or cable. Transmit and receive alternating signal lines may be carried on the same cables in other embodiments. The bias voltages may be generated elsewhere in the system and provided along the same cables, then separated from the AC signals at the transducer with electrical circuits, such as bias-T circuits.

Distinct alternating signal lines 20, 22 are provided for each electrode 14, 16 to be used in a transmit or receive time delay aperture. Alternatively, the alternating signal lines are used to implement a phase aperture, such as a Fresnel aperture. The transmit alternating signal lines 20 connect with a transmit beamformer (not shown). The transmit beamformer relatively delays and/or phase-shifts and apodizes signals from different channels. Each of the channels connects to a different one of the transmit alternating signal lines 20 and associated electrodes 14 or 16. The receive alternating signal lines 22 connect with a receive beamformer (not shown). The receive beamformer relatively delays and/or phase-shifts and apodizes signals on different channels. Each of the channels connects to a different one of the receive alternating signal lines 22 and associated electrodes 14 or 16. The data from the channels is summed together with appropriate time delays and/or phase shifts to isolate a spatial location. The transmit and receive beamformers operate as delay and/or phase based beamformers. The alternating signal lines 20, 22 connect with different electrodes 14, 16 to form the time delay aperture.

The alternating signal lines 20, 22 are connectable with the electrodes 14, 16. In the embodiment of FIG. 1, the arrangement of the high and low voltage switches 24 and 26 effectively connect and disconnect the alternating signal lines 20, 22 from the electrodes 14, 16. By grounding the transmit alternating signal line 20, the bias generator 18 connects to one of the electrodes 16 while the receive alternating signal line 22 connects to one of the electrodes 14. By grounding the receive alternating signal line 22, the bias generator 18 connects to one of the electrodes 14 while the transmit alternating signal line 20 connects to one of the electrodes 16. Alternatively, switches, a multiplexer, a diode network, mechanical or MEMS relays or other devices in a different arrangement switchably connect the alternating signal lines 20, 22 to the electrodes 14, 16.

The high voltage switch 24 is a high voltage FET or other switch operable with 100-200 volts. Other voltage levels and corresponding switches may be used. The high voltage switches 24 of multiple channels are discrete components or are integrated on a multiplexer with switches 24 for some or all of the channels.

The low voltage switch 26 is a low voltage FET, transistor or other switch operable with 1-20 volts. Other voltage levels and corresponding switches may be used. The low voltage switches 26 of multiple channels are discrete components or are integrated on a multiplexer with switches 26 for some or all of the channels.

The switches 24, 26 are operable to connect the bias generator 18 to one of the row electrodes 16 and the transmit alternating signal line 20 to one of the column electrodes 14 during transmit events. The high voltage switch 24 is open and the low voltage switch 26 is closed, grounding the vertical electrodes of the transducer. During transmit events, the bias generator 18 is connected to one of the column electrodes 14, and the transmit alternating signal line 20 is connected to one of the row electrodes 16. During receive, the low voltage switch 26 is open, and the high voltage switch 24 is closed, grounding the horizontal electrodes of the transducer.

After transmission, the switches 24, 26 are switched to change the bias and time delay apertures relative to the transducer 12. The switching rotates the aperture between transmit and receive, such as rotating a time delay aperture of the alternating signal lines 20, 22 of multiple channels and a Fresnel aperture of the bias generator 18 between transmit and receive responsive to the transmit. The switches 24, 26 alter between the transmit and receive alternating signal lines 20, 22 as part of switching the apertures. Alternatively, the same alternating signal lines 20, 22 are used for transmit and receive operation, so are used for both configurations of the switches 24, 26.

In the embodiment of FIG. 1, 2N electrodes 14, 16 are provided for the bias and alternating signal lines 20, 22. A grounding electrode may also be provided. The 2N electrodes are for N² elements. A total of 2N (or fewer, if the cables are muxed) cables for the alternating signal lines 20, 22 are provided for addressing all of the elements. For a non-square array, the apertures are interchanged by switching M+N lines, where M is the number of rows and N is the number of columns. The bias generator 18 is provided at the transducer 12 or in a probe, or in the ultrasound system. Alternatively, additional bias lines, such as seven, are provided and switches at the probe route the bias signals to form the Fresnel aperture. N amplifiers and 2N switches are located in the probe handle. In alternative embodiments, additional cables are provided, and/or additional or fewer components are provided in the probe.

The capacitors 28, 30 are grounding capacitors. The capacitor 28 connects between the high voltage switch 24 and the transmit alternating signal line 20. The capacitor 30 connects between the low voltage switch 26 and the bias generator 18. The grounding capacitors 28, 30 allow grounding of alternating signals while maintaining or allowing DC or bias signals (e.g., signals that vary at lower frequencies than the alternating signal frequencies).

In alternative embodiments, the elements of the transducer 12 are individually addressable without a matrix configuration. For example, a separate electrode connection is provided for each element. Multiplexers or other switches may be used to route the bias and alternating signals to different groups of elements.

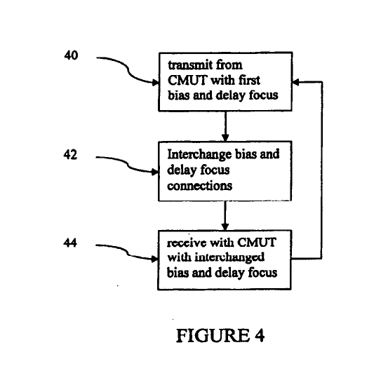

FIG. 4 shows one embodiment of a method for ultrasound imaging with a capacitive membrane ultrasound transducer. Additional, different or fewer acts may be provided in the method. The method uses the system of FIG. 1 or a different system.

The method of FIG. 4 includes transmitting and receiving acoustic energy with a cMUT, electrostrictive crystal transducer or other transducer using a bias for transducing between acoustic and electrical energies. The transmitting and receiving are responsive to bias signals applied to the ultrasound transducer and to alternating signals applied to or received from the transducer.

In act 40, acoustic energy is transmitted from the transducer. For the transmission, bias lines connect along a one direction on the transducer, and transmit signal lines connect along a different direction on the transducer. For example, the bias lines connect to azimuth extending electrodes spaced across an elevation dimension, and the transmit signal lines connect to elevation extending electrodes spaced across an azimuth dimension. The elevation and azimuth dimensions are orthogonal. The bias lines form a phase-shifted aperture across the elevation dimension, and the transmit signal lines form a time delay or phase aperture across the azimuth dimension. An opposite arrangement (e.g., phase-shifted bias aperture in the azimuth dimension and time delay or phase alternating signal aperture in the elevation dimension) may be provided for transmission.

The bias lines apply bias voltages to the transducer. The bias voltages may be associated with two or more levels. Different levels of bias are applied to different elements. In one embodiment, the bias voltages include positive and negative levels, allowing focusing with a Fresnel pattern. Three or more, such as five or seven, different bias levels are applied across the bias aperture. Where the bias aperture extends along the azimuth dimension, the Fresnel pattern focuses in azimuth. Where the bias aperture extends along the elevation dimension, the Fresnel pattern focuses in elevation.

The transmit signal lines connect a transmit beamformer to the transducer. The transmit beamformer applies a time delay (or phase-shifted) pattern to signals on the transmit signal lines connected with the transducer. Where the time delay aperture extends along the azimuth dimension, the time delay pattern focuses in azimuth. Where the time delay aperture extends along the elevation dimension, the time delay pattern focuses in elevation. In act 40, the time delay and bias apertures extend along different, such as orthogonal, directions.

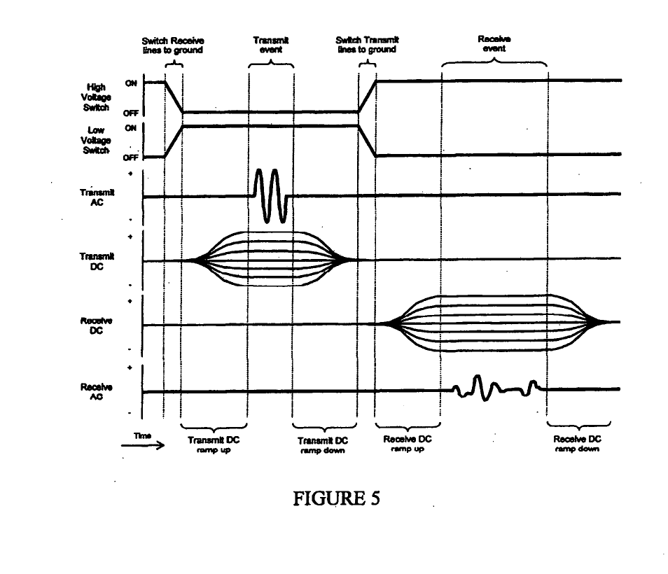

FIG. 5 shows the timing for a single channel. FIG. 5 shows a timing diagram for the method of FIG. 4 implemented with the system of FIG. 1. The high voltage switch is turned off for transmit. The low voltage switch is turned on for transmit. After switching, seven different levels of bias are applied along the bias aperture. The non-zero levels are gradually ramped up as represented by the transmit DC timing. Once the bias levels are ramped up (or while ramping is occurring), the transmit waveform is applied as represented by the transmit AC timing. Subsequently, the bias levels are gradually ramped down to zero values.

Referring again to FIG. 4, the bias and time delay apertures are interchanged or moved in act 42. Bias and delay focus connections are altered. Alternatively, only bias or only the time delay apertures are moved. The connections for the bias signals and alternating signals are interchanged or altered between a transmit event and a receive event responsive to the transmit event. The change rotates the acoustic aperture between the transmit event and the receive event responsive to the transmit event. For example, the acoustic aperture defined by the bias and alternating signal connections is rotated by about 90 degrees. Interchanging switches between apertures, while changing may move the apertures relative to the transducer with or without switching between the apertures

In one embodiment, the bias signals create a Fresnel aperture. Bias signals with at least five, seven or other number of different levels, including positive and negative levels, generate the Fresnel aperture. Altering the connections and applied position of the bias signals electrically rotates the Fresnel aperture. The alternating signals are for a time delay aperture. Altering the connections and applied position of the alternating signals electronically rotates the time delay aperture. By altering the location of the Fresnel and time delay aperture between transmit and receive events, round trip beam profiles are products of both a Fresnel and a time delay beam profile in both azimuth and elevation.

To increase the isotropic characteristic of received signals for volumetric imaging, the bias lines are connected orthogonal to signal lines on the transducer for transmit, and the bias lines are reconnected orthogonal to the signal lines at a different orientation on the transducer for receive. For example, the different bias signals are applied in an elevation pattern for the transmit event, and the alternating signals are delayed in an azimuth pattern for the transmit event. The different bias are applied in an azimuth pattern for a corresponding receive event, and the alternating signals are delayed in an elevation pattern for the receive event. The patterns used for transmit and receive may be the same or different.

FIG. 5 shows altering the position or status of the high and low voltage switches. The alternation rotates or changes the acoustic aperture position. For example, the bias and time delay apertures are interchanged.

Referring again to FIG. 4, acoustic energy is received with the transducer in act 44. The reception is responsive to the transmission in act 40. Due to the interchange or other alteration in act 42, the reception of act 44 is performed with the bias lines and receive signal lines connected along different directions than the bias lines and transmit signal lines for the transmission of act 40. The different directions for reception than for transmission are opposite (e.g., rotate aperture 90 degrees) or not opposite (e.g., rotate aperture other than 90 degrees or rotate bias connections differently than signal line connections). For example, the different directions are along the azimuth and elevation dimensions of the transducer array. During reception, the direction of the bias aperture and the time delay aperture are orthogonal, but another angle may be used. The orthogonal apertures are interchanged to rotate by 90 degrees for transmission.

In one embodiment, the bias signals create a Fresnel aperture. Bias signals with at least five different levels, including positive and negative levels, generate the Fresnel aperture. Signals received along another direction are delayed, forming a time delay aperture.

In FIG. 5, the low voltage switch is turned off for receive. The high voltage switch is turned on for receive. After switching, seven different levels of bias are applied along the bias aperture. A different DC timing line is provided to reflect the different connections and associated aperture. The non-zero levels are gradually ramped up as represented by the receive DC timing. Once the bias levels are ramped up (or while being ramped), the receive waveform is sampled or received as represented by the receive AC timing. Subsequently, the bias levels are gradually ramped down to zero values. Transmit bias lines may be ramped down while simultaneously being ramped up for receive.

The transmission act 40, interchange act 42 and reception act 44 are repeated for a same or different scan lines. For example, the acts 40, 42, 44 are repeated at least once for each of a plurality of scan lines in a volume. The resulting data represents the volume. Using rendering, such as surface rendering or ray line projections, a three-dimensional representation of the volume is generated. The viewing direction for the three-dimensional representation is manually or automatically selected and may change. By interchanging the Fresnel bias aperture and the time delay aperture between transmit and receive events, the three-dimensional representation is associated with sidelobe levels along one direction that are substantially the same as sidelobe levels along another direction, such as an orthogonal direction. Time delay focusing produces significantly lower side lobes than Fresnel phase shift focusing, leading to asymmetry. By electronically rotating the transducer aperture between transmit and receive within the interval of a single beam, the round trip beam profile is product of both time delay and Fresnel focusing. The round trip beam profile has low overall side lobes that are substantially isotropic in elevation and azimuth. In general, a transducer (cMUT or electrostrictive crystal) is patterned or segmented into one or more segments which are mechanically isolated from each other. A first set of electrodes, placed in the vicinity of the transducer, are aligned in a certain direction and are electrically connected to a set of groups of transducer segments. A second set of electrodes, also placed in the vicinity of the transducer, are aligned in a different direction from the first and are electrically connected to a different set of groups of transducer segments.

During transmit, alternating signals are applied to a subset of electrodes drawn from both the first and second sets, and smoothly varying bias voltages (having lower frequencies than the alternating signals or no variation at all) are applied to another subset of electrodes drawn from both sets. The two subsets of alternating signals and bias voltages may have many, few, or no shared elements. Bias voltages and alternating signals may be applied to the same electrodes simultaneously, or to different electrodes. Acoustic energy is generated by those segments that simultaneously see both a bias voltage and alternating signal in some combination. Time-delay based focusing will be brought about by either the alternating signal or by the smoothly varying bias, and phase-shift based focusing will be relegated to the other electrical mode. In this way, time-delay focusing and phase-shift focusing will occur along different axes in space.

During receive, the bias voltages are attached to a new subset of electrodes, different from the transmit subset and/or this same subset is used but the bias voltages are made to smoothly vary in a materially different way. At the same time, alternating signals are received from a new subset of electrodes and/or from the same subset but beamformed by the ultrasound system in a materially different way. During receive, the physical axes along which time-delay focusing and phase-shift focusing of the acoustic signal occur are reversed from the transmit case, leading to a more isotropic beam profile.

In one embodiment implementing the general approach above, row electrodes are arranged in strips on one side of a cMUT acoustic transducer which has been segmented into small 2D elements. Orthogonal column electrodes are arranged in strips on the opposite side.

During transmit, a constant bias voltage arranged in a Fresnel pattern is applied to the column electrodes, and alternating signals are applied to the row electrodes. Acoustic energy is generated in the region where bias voltage and alternating signals coincide. Time-delay focusing is brought about by the alternating signals and phase-shift focusing is brought about by the Fresnel bias pattern.

Directly after transmit, the bias voltage is removed from the row electrodes and these are subsequently grounded so they can no longer support alternating signals.

During receive, a smoothly varying bias voltage arranged in a dynamic Fresnel pattern is applied to the row electrodes, bringing about dynamic phase-shift focusing. At the same time, alternating signals are received from the column elements and are dynamically focused via time-delays. In this way, the physical axes of time-delay and phase-shift focusing have been switched between transmit and receive.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for ultrasound imaging with an ultrasound transducer responsive to a bias for transduction, the method comprising:
transmitting from the ultrasound transducer responsive to the bias for transduction with bias lines connected along a first direction of the ultrasound transducer and transmit signal lines connected along a second direction different than the first direction;
receiving, with the ultrasound transducer in response to the transmitting, with the bias lines connected along a direction different from the first direction and receive signal lines connected along a direction different from the second direction.

2. The method of claim 1 wherein receiving comprises receiving with the direction different from the first direction being the second direction and the direction different from the second direction being the first direction.

3. The method of claim 1 wherein transmitting comprises transmitting with the first direction orthogonal to the second direction and wherein receiving comprises receiving with the direction different from the first direction orthogonal to the direction different than the second direction.

4. The method of claim 1 wherein transmitting comprises transmitting from a multi-dimensional array of elements of the ultrasound transducer having first electrodes extending along a elevation dimension of the array and second electrodes extending along an azimuth dimension of the array, the first direction being one of the elevation or azimuth dimensions, the second direction being the other one of the elevation or azimuth dimensions, and wherein receiving comprises receiving with the direction different than the first direction is the other one of the elevation or azimuth dimensions and the direction different than the second direction is the one of the elevation or azimuth dimensions.

5. The method of claim 1 wherein transmitting comprises:
applying a first Fresnel pattern to the ultrasound transducer with the bias lines, the first Fresnel pattern being along the first direction; and
applying a first time delay pattern to signals on the transmit signal lines connected with the ultrasound transducer, the first time delay pattern being along the second direction; and
wherein receiving comprises:
applying a second Fresnel pattern to the ultrasound transducer with the bias lines, the second Fresnel pattern being along the direction different than the first direction; and
applying a second time delay pattern to signals from the ultrasound transducer, the second time delay pattern being along the direction different from the second direction;

6. The method of claim 1 wherein transmitting comprises applying a first Fresnel pattern to the ultrasound transducer with the bias lines, the first Fresnel pattern being along the first direction and responsive to at least five different bias levels; and wherein receiving comprises applying a second Fresnel pattern to the ultrasound transducer with the bias lines, the second Fresnel pattern being along the second direction and responsive to the at least five different bias levels.

7. The method of claim 1 further comprising:
repeating the transmitting and receiving along a plurality of scan lines in a volume; and
generating a three-dimensional representation of the volume;
wherein the three-dimensional representation is associated with sidelobe levels along the first direction substantially a same as sidelobe levels along the second direction.

8. In a method for transmitting and receiving acoustic energy with a ultrasound transducer responsive to a bias for operation, the transmitting and receiving being responsive to bias signals applied to the ultrasound transducer and to alternating signals, an improvement comprising:
interchanging the bias signals and alternating signals between a transmit event and a receive event responsive to the transmit event.

9. The improvement of claim 8 wherein interchanging comprises rotating an acoustic aperture between the transmit event and the receive event responsive to the transmit event.

10. The improvement of claim 9 wherein rotating comprises rotating the acoustic aperture by about 90 degrees.

11. The improvement of claim 8 wherein interchanging comprises connecting bias lines orthogonal to signal lines on the ultrasound transducer, and reconnecting the bias lines orthogonal to the signal lines at a different orientation on the ultrasound transducer.

12. The improvement of claim 8 wherein interchanging comprises:
applying different bias signals in a first elevation pattern for the transmit event;
delaying the alternating signals in a first azimuth pattern for the transmit event;
applying the different bias signals in a second azimuth pattern for the receive event; and
delaying the alternating signals in a second elevation pattern for the receive event.

13. The improvement of claim 8 wherein interchanging comprises electrically rotating a Fresnel aperture responsive to the bias signals and a delay aperture responsive to the alternating signals.

14. The improvement of claim 13 further comprising:
generating the Fresnel aperture as a function of the bias signals with at least five different levels.

15. The improvement of claim 8 wherein the ultrasound transducer is a multidimensional array of M elements with rows of first electrodes extending along elevation and columns of second electrodes extending along azimuth, wherein interchanging comprises switching with about two times a square root of M bias and signal lines.

16. The improvement of claim 8 wherein interchanging comprises providing round trip beam profiles in both azimuth and elevation which are products of both a Fresnel and a time delay beam profile.

17. A system for ultrasound imaging with an ultrasound transducer operable with bias, the system comprising:
first electrodes on the ultrasound transducer operable with bias, the first electrodes distributed across a second direction and each extending over multiple elements along a first direction;
second electrodes on the ultrasound transducer, the second electrodes distributed across the first direction and each extending over multiple elements along the second direction;
a bias generator connectable with the first and second electrodes;
alternating signal lines connectable with the first and second electrodes; and
at least one switch operable to connect the bias generator to the first electrodes during transmit and the second electrodes during receive and operable to connect the alternating signal lines to the second electrodes during transmit and the first electrodes during receive.

18. The system of claim 17 wherein the alternating signal lines connect with a delay beamformer.

19. The system of claim 17 wherein the alternating signal lines comprise transmit lines connected with a transmit beamformer and receive lines connected with a receive beamformer, the transmit lines separate from the receive lines, the at least one switch operable to connect the transmit lines as the alternating signal lines during transmit and the receive lines as the alternating signal lines during receive.

20. The system of claim 17 wherein the first direction comprises an elevation direction and the second direction comprises an azimuth direction orthogonal to the elevation direction, the at least one switch operable to rotate an aperture between transmit and receive.

21. The system of claim 17 wherein the bias generator is operable to generate at least five bias levels, different ones of the bias levels applied to different ones of the first and second electrodes during transmit and receive, respectively, the different ones of the bias levels comprises a Fresnel aperture.

22. The system of claim 17 wherein the at least one switch is operable to rotate a time delay focus aperture of the alternating signal lines and a Fresnel aperture of the bias generator between transmit and receive responsive to the transmit.

23. The system of claim 19 wherein the at least one switch comprises high voltage switches connected with the transmit lines and low voltage switches connected with the receive lines, the bias generator connected between the transmit and receive lines; and
further comprising first grounding capacitors connected between the high voltage switches and the transmit lines and second grounding capacitors connected between the low voltage switches and the bias generator.

24. The system of claim 17 wherein the bias generator is operable to dynamically focus during receive operation as a function of bias signals.

25. The method of claim 1 wherein receiving comprises dynamically focusing with the bias lines.
